(19) United States
(12) Patent Application Publication
(10) Pub. No.: US 2026/0334925 A1
(43) Pub. Date: Oct. 29, 2026

(54) SUCTION TISSUE ACQUISITION AND SELF-RETRACTING STYLET

(71) Applicant: Devicor Medical Products, Inc., Cincinnati, OH (US)

(72) Inventors: Andrew P. NOCK, Dayton, OH (US); Maria B. ROMA, Cincinnati, OH (US); Kyle WAGNER, Mason, OH (US); Jack RANDALL, Cincinnati, OH (US); Lyle JAMES, Kettering, OH (US)

(21) Appl. No.: 19/703,523

(22) Filed: Jun. 10, 2026

Related U.S. Application Data
(63) Continuation of application No. PCT/US2024/059194, filed on Dec. 9, 2024.
(60) Provisional application No. 63/615,875, filed on Dec. 29, 2023.

(51) Int. Cl. A61B 10/02

(52) U.S. Cl. CPC A61B 10/0275

(57) ABSTRACT

A core needle biopsy device includes a body, a needle assembly and a drive assembly. The needle assembly extends distally from the body and includes a piercer, a hollow outer cannula, and a cutting element. The outer cannula defines an internal lumen. The piercer is disposed within the outer cannula. The cutting element extends along an outer surface or an inner surface of the outer cannula. The cutting element is configured to translate relative to the outer cannula. The drive assembly is configured to selectively move one or more of the piercer, the outer cannula, and the cutting element. The drive assembly is further configured to move the piercer relative to the outer cannula to open and close the internal lumen defined by the outer cannula.

### PRIORITY

This application claims priority to U.S. Provisional Application No. 63/615,875, entitled “Suction Tissue Acquisition and Self-Retracting Stylet,” filed on Dec. 29, 2023, the disclosure of which is hereby incorporated by reference herein.

### BACKGROUND

Biopsy samples have been obtained in a variety of ways in various medical procedures including open and percutaneous methods using a variety of devices. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device.

One technique for collecting a breast biopsy is to use a core needle biopsy device. Core needle biopsy devices frequently use a sharp, solid piercer equipped with a lateral tissue receiving notch positioned adjacent to the distal end of the piercer. When tissue is received within the notch, an elongate hollow cutting sheath is translated over the notch to sever a tissue sample. The severed tissue sample is then stored within the notch until both the piercer and the cutting sheath are removed from the patient. Thus, in core-needle biopsy devices, only one tissue sample can be collected per insertion of the piercer and cutting sheath.

Another technique for conducting a breast biopsy is to collect a breast biopsy using a vacuum-assisted breast biopsy device. In contrast to core needle breast biopsy procedures, vacuum-assisted breast biopsy devices permit the biopsy device to remove multiple samples without requiring removal of the device from the breast after every sample is collected. For instance, in a vacuum assisted breast biopsy device, a hollow needle is used to penetrate tissue. The hollow needle includes a lateral aperture adjacent to a sharp distal tip. A hollow cutter is disposed within the hollow needle and is moved axially relative to the lateral aperture of the needle to sever tissue samples. Once a tissue sample is severed by the hollow cutter, the tissue sample is transported axially though the cutter and collected in a tissue collection feature.

Examples of vacuum assisted biopsy devices and biopsy system components are disclosed in U.S. Pat. No. 5,526,822, entitled “Method and Apparatus for Automated Biopsy and Collection of Soft Tissue,” issued Jun. 18, 1996; U.S. Pat. No. 6,086,544, entitled “Control Apparatus for an Automated Surgical Biopsy Device,” issued Jul. 11, 2000; U.S. Pat. No. 7,442,171, entitled “Remote Thumbwheel for a Surgical Biopsy Device,” issued Oct. 8, 2008; U.S. Pat. No. 7,854,706, entitled “Clutch and Valving System for Tetherless Biopsy Device,” issued Dec. 1, 2010; U.S. Pat. No. 7,938,786, entitled “Vacuum Timing Algorithm for Biopsy Device,” issued May 10, 2011; U.S. Pat. No. 8,118,755, entitled “Biopsy Sample Storage,” issued Feb. 1, 2012; and U.S. Pat. No. 8,206,316, entitled “Tetherless Biopsy Device with Reusable Portion,” issued on Jun. 26, 2012. The disclosure of each of the above-cited U.S. Patents is incorporated by reference herein.

Examples of core needle biopsy devices are disclosed in U.S. Pat. No. 5,560,373, entitled “Needle Core Biopsy Instrument with Durable or Disposable Cannula Assembly,” issued on Oct. 1, 1996; U.S. Pat. No. 5,817,033, entitled “Needle Core Biopsy Device,” issued on Oct. 6, 1998; and U.S. Pat. No. 5,511,556, entitled “Needle Core Biopsy Instrument,” issued on Apr. 30, 1996. The disclosure of each of the above-cited U.S. Patents is incorporated by reference herein.

In some circumstances, it may be desirable to combine aspects of core-needle biopsy devices with aspects of vacuum-assisted biopsy devices to achieve benefits of both devices without one or more drawbacks associated with one device or the other. For instance, core-needle biopsy devices in some circumstances may provide simplicity and ease of use relative to vacuum assisted biopsy device. Meanwhile, in other circumstances, vacuum assisted biopsy devices may provide greater accuracy and procedure efficiency relative to core-needle biopsy devices. It may therefore be desirable to combine aspects of both devices to achieve one or more benefits of both devices.

While several systems and methods have been made and used for obtaining and processing a biopsy sample, it is believed that no one prior to the inventor has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a perspective view of an illustrative biopsy device;

FIG. 2 depicts a detailed perspective view of a needle assembly of the biopsy device of FIG. 1;

FIG. 3 depicts a detailed perspective view of a piercer of the needle assembly of FIG. 2;

FIG. 4A depicts a side cross-sectional view of the needle assembly of FIG. 2, the cross-section taken along line 4-4 of FIG. 2;

FIG. 4B depicts another side cross-sectional view of the needle assembly of FIG. 2, the piercer of FIG. 3 being retracted relative to an outer cannula of the needle assembly;

FIG. 4C depicts yet another side cross-sectional view of the needle assembly of FIG. 2, the needle assembly severing a tissue sample;

FIG. 5 depicts a perspective view of an illustrative alternative needle assembly that may be readily incorporated into the biopsy device of FIG. 1;

FIG. 6 depicts a side cross-sectional view of the needle assembly of FIG. 5, the cross-section taken along line 6-6 of FIG. 5 and a piercer of the needle assembly being retracted relative to an outer cannula of the needle assembly;

FIG. 7A depicts another side cross-sectional view of the needle assembly of FIG. 5, the piercer being advanced relative to the outer cannula to penetrate tissue;

FIG. 7B depicts yet another side cross-sectional view of the needle assembly of FIG. 5, the piercer being retracted relative to the outer cannula to receive tissue within the outer cannula;

FIG. 7C depicts still another side cross-sectional view of the needle assembly of FIG. 5, a cutting element of the needle assembly being moved to sever a tissue sample within the outer cannula; and

FIG. 7D depicts still another side cross-sectional view of the needle assembly of FIG. 5, the tissue sample of FIG. 7C being transported through the needle assembly.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

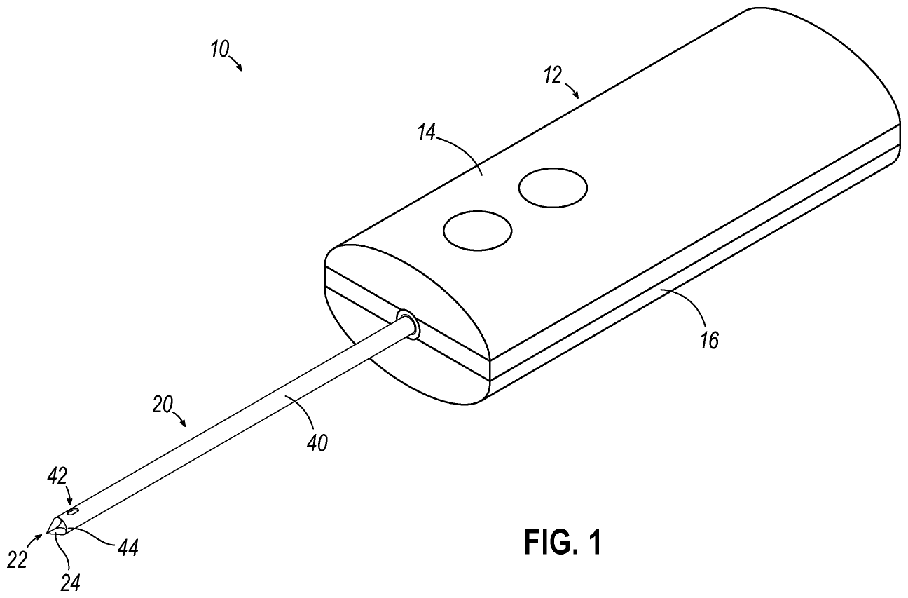

FIG. 1 shows an illustrative biopsy device (10) for use in a breast biopsy procedure. Biopsy device (10) of the present example includes a body (12) and a needle assembly (20) extending distally from body (12). Body (12) includes a holster housing (14) and a probe housing (16). As will be described in greater detail below, holster housing (14) and probe housing (16) enclose various components of biopsy device (10), which are used to drive needle assembly (20) through a cutting cycle and a tissue acquisition cycle. For instance, in some examples holster housing (14) can enclose more expensive or durable parts such that holster housing (14) can be reused. Similarly, probe housing (16) can enclose less expensive or less durable parts such that probe housing (16) (along with needle assembly (20)) can be disposable. When coupled together, holster housing (14) and probe housing (16) of the present example are configured to couple together such that body (12) is sized and shaped for grasping by an operator using a single hand. Although the terms “holster” and “probe” used herein may imply that one part receives another part, it should be understood that no such limitation is intended. For instance, in some examples certain components of holster housing (14) can be received within probe housing (16).

In some examples, biopsy device (10) may further includes a tissue sample holder (not shown). Such tissue sample holders may be disposed at the distal end of body (12) or other components thereof. Such tissue sample holders may include a variety of configurations. For instance, in one configuration, the tissue sample holder is generally configured as a hollow compartment that can receive tissue samples severed by needle assembly (20). In other examples, the tissue sample holder can include a basket to hold the collected tissue samples and/or strain fluids from the severed tissue samples. In still other examples, such a basket can be segregated into different compartments to store tissue samples in an organized configuration. In such examples, the basket can be movable to aid in depositing severed tissue samples into a particular compartment. Additionally, it should be understood that in some examples the tissue sample holder is in communication with a vacuum source to aid in transporting tissue samples to the tissue sample holder. Of course, various other alternative configurations of the tissue sample holder will be apparent to those of ordinary skill in the art in view of the teachings herein.

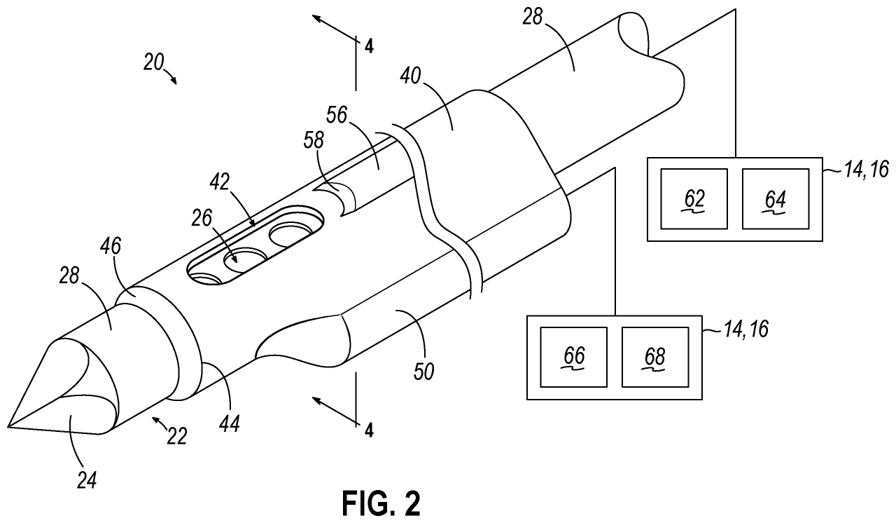

FIG. 2 shows needle assembly (20) in greater detail. As can be seen, needle assembly (20) comprises an elongate piercer (22) and an elongate outer cannula (40). As will be described in greater detail below, piercer (22) is generally movable relative to outer cannula (40) to pierce tissue and collect tissue samples, while outer cannula (40) is generally fixed or movable relative to piercer (22) to facilitate receipt of one or more tissue samples within a lateral aperture (42) defined by outer cannula (40).

Both piercer (22) and outer cannula (40) are optionally in communication with one or more features of body (12), which are contained within holster housing (14) or probe housing (16). For instance, in the present example, piercer (22) is in communication with one or more piercer drive components (62) and/or one or more piercer fluid sources (64). Similarly, outer cannula (40) is in communication with one or more cannula drive components (66) and/or one or more cannula fluid sources (66).

Drive components (62, 66) are generally configured to drive physical movement of piercer (22), outer cannula (40), and/or other components of biopsy device (10) relative to body (12). Although drive components (62, 66) are shown separately in the present example, it should be understood that in other examples, drive components (62, 66) are interconnected or can be configured as a single drive component with separate outputs for piercer (22) and outer cannula (40). In some examples, drive components (62, 66) are configured as manually driven mechanisms with latches, buttons, levers, and/or etc. Such manually driven mechanisms are configured to permit drive of piercer (22), outer cannula (40), or other components by hand. In other examples, drive components (62, 66) are motor driven and can be controlled automatically or semi-automatically by various electromechanical control features.

Suitable movements driven by drive components (62, 66) include, for example, firing of piercer (22) and/or outer cannula (40). Other suitable movements driven by drive components (62, 66) include, for example, precise axial and/or lateral movements of piercer (22) and/or outer cannula (40) relative to each other to facilitate severing of tissue samples as will be described in greater detail below. In some examples, suitable movements driven by drive components (62, 66) also includes, for example, rotation of piercer (22) and/or outer cannula (40). In addition, or in the alternative, suitable movements driven by drive components (62, 66) includes vibration of piercer (22) and/or outer cannula (40). Vibration of components such as outer cannula (40) may be desirable in some examples to facilitate penetration of tissue. Of course, other suitable movements may be driven by drive components (62, 66) as will be apparent to those of ordinary skill in the art in view of the teachings herein.

Fluid sources (64, 68) are generally configured to communicate one or more fluid mediums to piercer (22) and/or outer cannula (40). Suitable fluid mediums include, for example, vacuum, atmospheric air, saline, saline in combination with therapeutic agents, therapeutic agents, and/or etc. As will be described in greater detail below, such fluid mediums can be communicated through a hollow interior portion of piercer (22) and/or outer cannula (40) to facilitate the collection of one or more tissue samples using piercer (22) and outer cannula (40). Although not shown, it should be understood that fluid sources (64, 68) can include components to facilitate fluid communication such as motors, reservoirs, containers, pumps, and/or etc. Additionally, although fluid sources (64, 68) are shown separately herein, it should be understood that fluid sources (64, 68) are combined into a single component with multiple outputs in some examples.

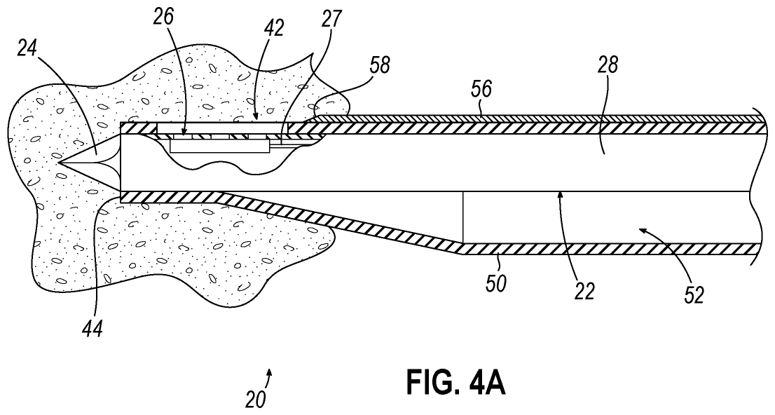

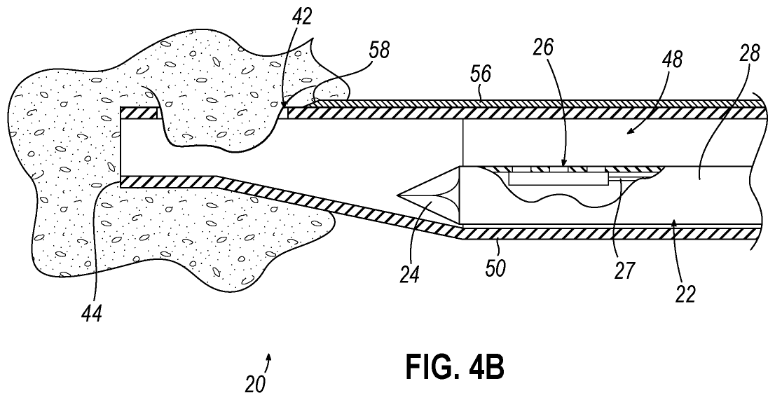

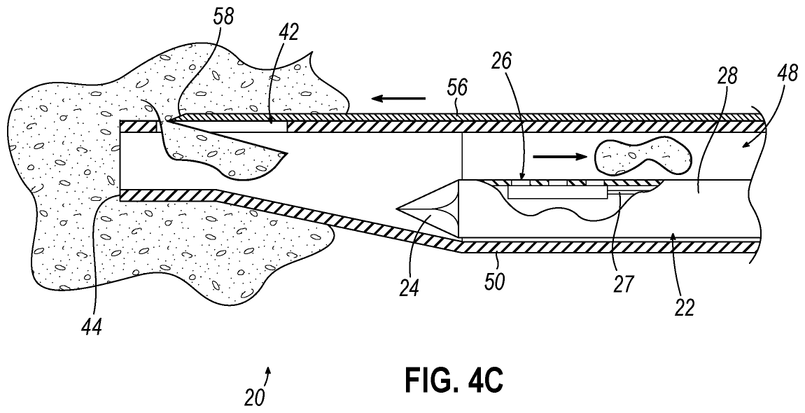

Outer cannula (40) includes an elongate cylindrical structure defining an internal lumen (48) (see FIG. 4A), a distal end (44) and a projection (50) disposed proximally of distal end (44). Distal end (44) optionally includes a tapered edge (46) that is generally configured to provide a smooth interface between outer cannula (40) and piercer (22) as will be described in greater detail below. Although tapered edge (46) is generally oriented perpendicularly relative to a longitudinal axis defined by outer cannula (40), tapered edge (46) may be oriented at an angle relative to the longitudinal axis in other examples. In other examples, tapered edge (46) is omitted and distal end (44) includes a blunt configuration. In still other examples, tapered edge (46) can be sharpened in addition to, or in lieu of, being tapered. As will also be described in greater detail below, distal end (44) is generally of an open configuration such at least a portion of piercer (22) may extend through distal end (44) to protrude from outer cannula (40).

As described above, outer cannula (40) defines a lateral aperture (42). Lateral aperture (42) is disposed proximate distal end (44). As will be described in greater detail below, lateral aperture (42) is configured to receive tissue such that tissue can be received within internal lumen (48) of outer cannula (40). As will also be described in greater detail below, tissue received within internal lumen (48) can be severed to form a tissue sample with one or more components associated with outer cannula (40). As will be appreciated, lateral aperture (42) can be omitted in some examples with tissue being received and severed by other portions of outer cannula (40).

Projection (50) is generally configured as a portion of outer cannula (40) with an expanded diameter. In other words, projection (50) is configured as a bulbous section of outer cannula (40) that originates proximally of distal end (44) and extends proximally and axially the length of outer cannula (40). In the present example, projection (50) is oriented on a side of outer cannula (40) opposite lateral aperture (42). Projection (50) defines an internal recess (52) (see FIG. 4A) within the otherwise cylindrical structure of outer cannula (40) that is configured to receive at least a portion of piercer (22). As will be described in greater detail below, receipt of piercer (22) within internal recess (52) is configured to facilitate transport of one or more tissue samples through internal lumen (48) outer cannula (40). In other words, piercer (22) is configured to move between internal lumen (48) and internal recess (52) to selectively open and close internal lumen (48).

Needle assembly (20) further includes a cutting element (56) associated with outer cannula (40). Cutting element (56) is generally configured to move relative to lateral aperture (42) of outer cannula (40) to sever tissue disposed within lateral aperture (42). In the present example, cutting element (56) is formed as an elongate structure similar to a curved blade resting on an outer surface of outer cannula (40). In some examples, a portion of cutting element (56) is curved to correspond to the curvature of outer cannula (40). To facilitate cutting of tissue, the distal end of cutting element (56) includes a sharpened distal end (58). It should be understood that sharpened distal end (58) can define a variety of different shapes such as generally straight or curved (convex or concave). In addition, or in the alternative, sharpened distal end (58) can be angled relative to the longitudinal axis of cutting element (56) in some examples.

In the present example, cutting element (56) is disposed on the exterior of outer cannula (40). Although not shown, it should be understood that outer cannula (40) can include one or more fastening features configured to couple cutting element (56) to the exterior of outer cannula (40), while also permitting translation of cutting element (56) along the longitudinal axis of outer cannula (40). For instance, suitable fastening features can include tabs, channels, ledges, overhangs, pockets, and/or etc. In other examples, cutting element (56) is disposed within the interior of outer cannula (40) with similar fastening features included within outer cannula (40).

Cutting element (56) extends from lateral aperture (42) proximally for substantially the length of outer cannula (40). Such an extension may be desirable to permit movement of cutting element (56) to be driven by one or more of cannula drive components (66), piercer drive components (62), and/or separate dedicated drive components. In other examples, cutting element (56) extends for only a portion of the length of outer cannula (40). In such examples, one or more additional components such as arms and/or linkages can be used to facilitate transfer of movement from any suitable drive components to cutting element (56).

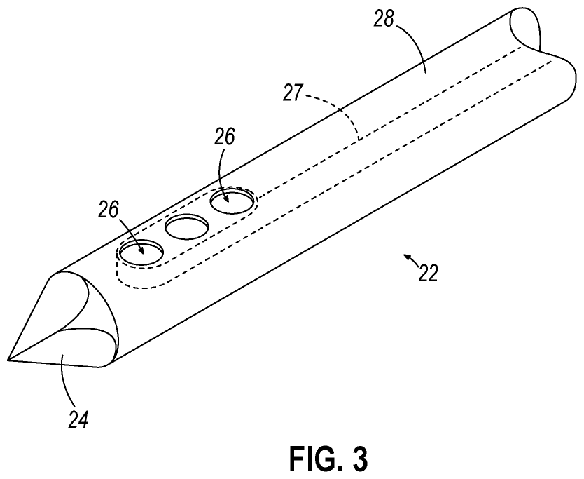

As best seen in FIG. 3, piercer (22) includes a generally hollow cylindrical wall (28) having a sharp distal tip (24). Cylindrical wall (28) defines a lumen (27) extending through the length of piercer (22) and one or more fluid openings (26) in communication with lumen (27). As will be described in greater detail below, distal tip (24) is generally configured to penetrate tissue of a patient when protruding from distal end (44) of outer cannula (40). As will also be described in greater detail below, lumen (27) and fluid openings (26) are generally configured to communicate fluid from piercer fluid source (64) into the interior of outer cannula (40). Such fluid communication may be desirable in some examples to facilitate collection and/or transport of one or more tissue samples via piercer (22) and outer cannula (40).

In some examples of biopsy device (10), dead space may impact operability. Dead space may be characterized in some circumstances as the length between a sample acquisition area and a needle, piercer, or stylet tip. In devices having a relatively large dead space, access to tissue proximate sensitive anatomical structures may be limited. For instance, in procedures where a suspicious lesion is near the chest wall, the space between the suspicious lesion and the chest wall may be limited. Consequently, limiting dead space may be desirable in this circumstance to permit access to the suspicious lesion while also avoiding contact with the chest wall.

Needle assembly (20) of the present example is generally configured to provide variable dead space at various stages of a biopsy procedure to thereby permit access to suspicious lesions proximate sensitive anatomy. Generally, piercer (22) is configured to selectively move relative to outer cannula (40) to adjust or entirely eliminate the dead space of needle assembly (20) as defined by the distance between the end of distal tip (24) and the distal end of lateral aperture (42). Consequently, piercer (22) can project from outer cannula (40) at some stages of a biopsy procedure and be retracted into outer cannula (40) at other stages of the biopsy procedure to provide variable dead space.

FIGS. 4A through 4C show an example of how such variable dead space can be used during a biopsy procedure. As best seen in FIG. 4A, piercer (22) is initially positioned relative to outer cannula (40) with distal tip (24) projecting from distal end (44) of outer cannula (40). In this configuration, the dead space of needle assembly (20) is generally increased relative to other configurations. As can be seen, while in this configuration, needle assembly (20) is used to penetrate tissue to position lateral aperture (42) relatively close to a suspicious lesion. In other words, this configuration is used for course positioning of needle assembly (20). Optionally, in some uses, guidance of needle assembly (20) during penetration of tissue can be facilitated using various image guidance mechanisms such as x-ray guidance, ultrasound guidance, and/or optical coherence tomography (OCT) guidance.

As can be seen in FIG. 4A, cutting element (56) is positioned in the open position relative to lateral aperture (42) during penetration of tissue. Tissue may be prevented from prolapsing into lateral aperture (42) during this stage due to the presence of piercer (22) adjacent to lateral aperture (42). In other uses, cutting element (56) is advanced relative to lateral aperture (42) to close lateral aperture (42). In such uses, cutting element (56) being in the closed position may be desirable to control the relationship between lateral aperture (42) and tissue prolapse regardless of the particular position of piercer (22) within outer cannula (40).

After needle assembly (20) is positioned as described above with distal tip (24) of piercer (22) projecting from outer cannula (40), piercer (22) is retracted to reduce the dead space of needle assembly (20). As best seen in FIG. 4B, piercer (22) is retracted into the interior of outer cannula (40). In this position, piercer (22) is positioned laterally of internal lumen (48) and into internal recess (52) of projection (50). In the present example, such movement of piercer (22) is facilitated by piercer drive components (62) cannula drive components (66), or a combination thereof. Due to the decreased dead space in this configuration, needle assembly (20) is optionally positioned at this stage to position lateral aperture (42) closer to a suspicious lesion. In other words, this configuration is optionally used for fine positioning of needle assembly (20).

Once needle assembly (20) is positioned within tissue as desired, needle assembly (20) can be used to sever a tissue sample as shown in FIGS. 4B and 4C. To sever a tissue sample, cutting element (56) is first retracted (if not already) relative to lateral aperture (42) to open lateral aperture (42). Once lateral aperture (42) is in the open configuration, tissue can be prolapsed though lateral aperture (42) into internal lumen (48) of outer cannula (40). In some uses, the prolapsing of tissue is facilitated using vacuum communicated through internal lumen (48) of outer cannula (40) via cannula fluid sources (68). A tissue sample can then be severed by advancing cutting element (56) distally relative to lateral aperture (42) as shown in FIG. 4C.

After the tissue sample is severed, the tissue sample is transported through outer cannula (40) as shown in FIG. 4C. As can be seen, vacuum is communicated through internal lumen (48) of outer cannula (40) to pull the tissue sample through internal lumen (48). As the tissue sample progresses through internal lumen (48), piercer (22) can communicate atmospheric air or pressurized fluid from piercer fluid sources (64) via lumen (27) and fluid openings (26). Such atmospheric air or pressurized fluid may be desirable to maintain a pressure imbalance between the proximal side of the tissue sample and the distal side of the tissue sample, thereby promoting transport of the tissue sample through internal lumen (48).

After the tissue sample is transported through outer cannula (40), the tissue sample can be deposited in a tissue sample holder (not shown) or other tissue collection area. The process described above with respect to FIGS. 4B through 4C can then be repeated without removal of needle assembly (20) from the patient to collect additional tissue samples until a desired number of tissue samples have been collected. Once a desired number of tissue samples are collected, needle assembly (20) can be removed from the patient.

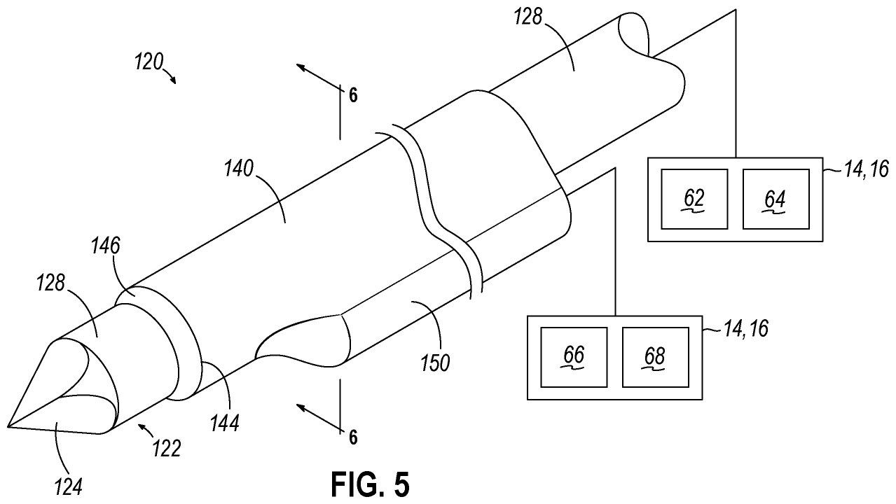

FIG. 5 shows an illustrative alternative needle assembly (120) that may be readily incorporated into biopsy device (10) described above in lieu of needle assembly (20) also described above. Needle assembly (120) of the present example is substantially similar to needle assembly (20) described above except where otherwise described herein. For instance, needle assembly (120) comprises an elongate piercer (122) and an elongate outer cannula (140). As similarly described above, piercer (122) is generally movable relative to outer cannula (140) to pierce tissue and collect tissue samples. However, unlike outer cannula (40) described above, outer cannula (140) of the present example omits structures similar to lateral aperture (42), as will be described in greater detail below.

As with piercer (22) and outer cannula (40) described above, both piercer (122) and outer cannula (140) are optionally in communication with one or more features of body (12), which are contained within holster housing (14) or probe housing (16). For instance, in the present example, piercer (122) is in communication with one or more piercer drive components (62) and/or one or more piercer fluid sources (64). Similarly, outer cannula (140) is in communication with one or more cannula drive components (66) and/or one or more cannula fluid sources (66).

As similarly described above, drive components (62, 66) are generally configured to drive physical movement of piercer (122) and outer cannula (140) respectively relative to body (12). Suitable movements driven by drive components (62, 66) include, for example, firing of piercer (122) and/or outer cannula (140). Other suitable movements driven by drive components (62, 66) include, for example, precise axial and/or lateral movements of piercer (122) and/or outer cannula (140) relative to each other to facilitate severing of tissue samples as will be described in greater detail below. In some examples, suitable movements driven by drive components (62, 66) also includes, for example, rotation of piercer (122) and/or outer cannula (140). Of course, other suitable movements may be driven by drive components (62, 66) as will be apparent to those of ordinary skill in the art in view of the teachings herein.

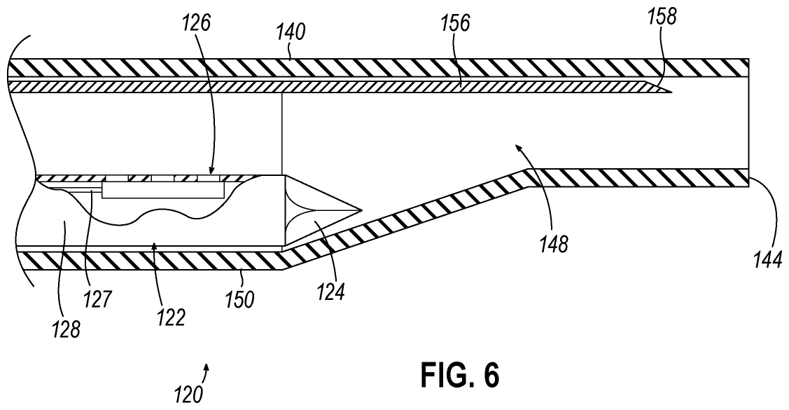

Outer cannula (140) includes an elongate cylindrical structure defining an internal lumen (148) (see FIG. 6), a distal end (144) and a projection (150) disposed proximally of distal end (144). Distal end (144) optionally includes a tapered edge (146) configured to provide a smooth transition between outer cannula (140) and a portion of piercer (22), as will be described in greater detail below. Although tapered edge (146) is generally oriented perpendicularly relative to a longitudinal axis defined by outer cannula (140), tapered edge (146) may be oriented at an angle relative to the longitudinal axis in other examples. In other examples, tapered edge (146) is omitted and distal end (144) includes a blunt configuration. In still other examples, tapered edge (146) can be sharpened in addition to, or in lieu of, being tapered.

Like projection (50) described above, projection (150) of the present example is generally configured as a portion of outer cannula (140) with an expanded diameter that originates proximally of distal end (144) and extends proximally and axially the length of outer cannula (140). Projection (150) defines an internal recess (152) (see FIG. 6) within the otherwise cylindrical structure of outer cannula (140) that is configured to receive at least a portion of piercer (122). As will be described in greater detail below, receipt of piercer (122) within internal recess (152) is configured to facilitate transport of one or more tissue samples through internal lumen (148) outer cannula (140). In other words, piercer (122) is configured to move between internal lumen (148) and internal recess (152) to selectively open and close internal lumen (148).

As best seen in FIG. 6, needle assembly (120) further includes a cutting element (156) associated with outer cannula (140). Similar to cutting element (56) described above, cutting element (156) of the present example is generally configured to move relative to a portion of outer cannula (140) to sever one or more tissue samples. Cutting element (156) is similarly formed as an elongate structure similar to a curved blade that is complementary to the shape of outer cannula (140). Additionally, to facilitate cutting of tissue, the distal end of cutting element (156) includes a sharpened distal end (158). It should be understood that sharpened distal end (158) can define a variety of different shapes such as generally straight or curved (convex or concave). In addition, or in the alternative, sharpened distal end (158) can be angled relative to the longitudinal axis of cutting element (156) in some examples.

Unlike the configuration described above with respect to cutting element (56) and outer cannula (40), cutting element (156) of the present example is disposed within the interior of outer cannula (140). Although not shown, it should be understood that outer cannula (140) can include one or more fastening features configured to couple cutting element (156) to the interior of outer cannula (140), while also permitting translation of cutting element (156) along the longitudinal axis of outer cannula (140). As similarly described above, suitable fastening features can include tabs, channels, ledges, overhangs, pockets, and/or etc.

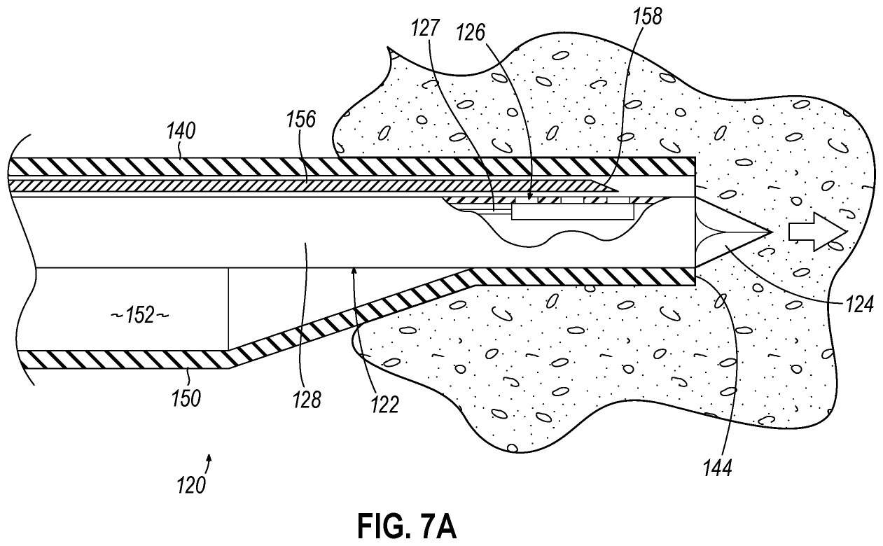

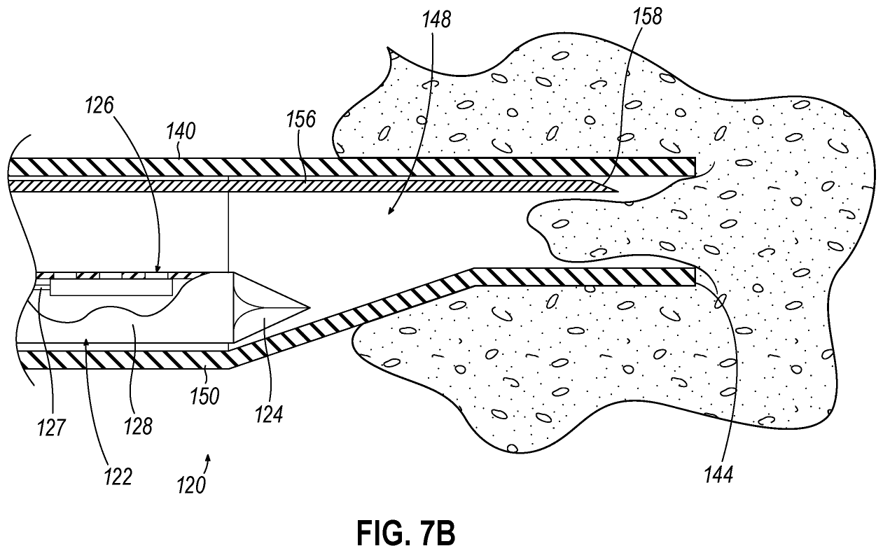

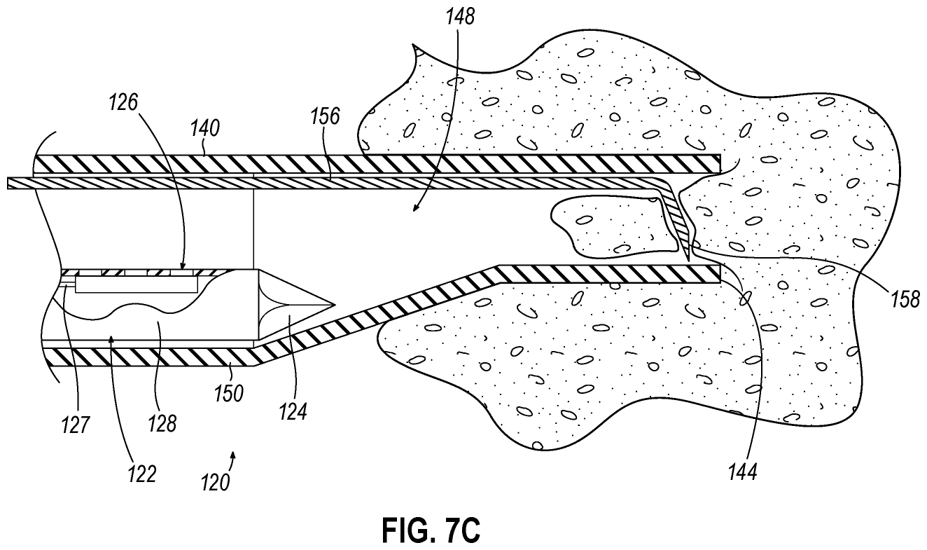

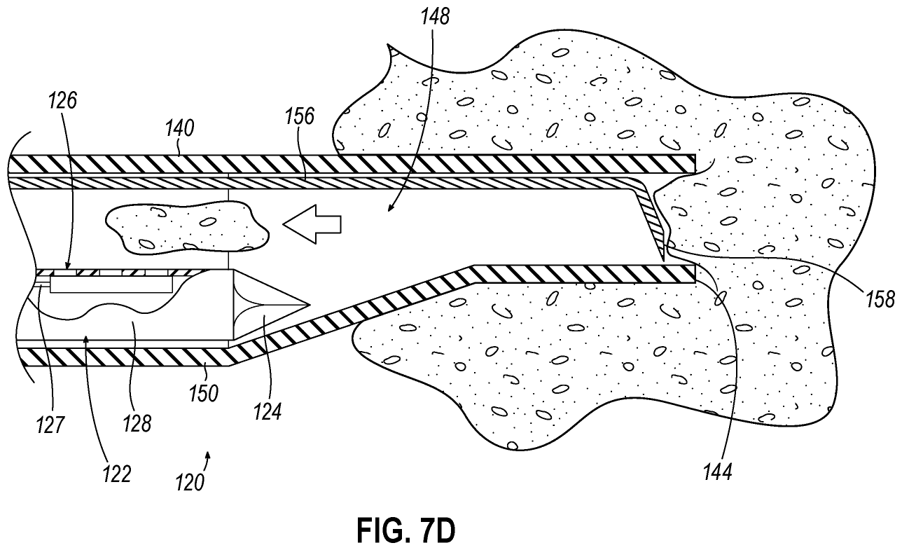

Unlike cutting element (56) described above, cutting element (156) of the present example is configured to bend or change in shape to facilitate cutting of tissue (see FIG. 7C). In other words, cutting element (156) is configured to remain straight when retracted within outer cannula (140), but bend laterally away from the interior of outer cannula (140) at a predetermined location during advancement of cutting element (156) relative to outer cannula (140). Thus, at least a portion of cutting element (156) is formed as a generally flexible material. As will be described in greater detail below, such bending may be desirable to facilitate a lateral cutting action similar to a guillotine. Although not shown, it should be understood that cutting element (156) is configured to cooperate with other features of outer cannula (140) or cutting element (156) itself to facilitate such bending. Such features may include, for example, ramps, detents, channels, and/or etc. In other examples, cutting element (156) can be configured in a variety of ways to achieve a similar cutting action in a different form. Suitable alternative forms may include, for example, one or more contracting elements, one or more collapsing elements similar to an iris mechanism, and/or one or more wire snares.

Cutting element (156) extends within outer cannula (140) proximally for substantially the length of outer cannula (140). Such an extension may be desirable to permit movement of cutting element (156) to be driven by one or more of cannula drive components (66), piercer drive components (62), and/or separate dedicated drive components. In other examples, cutting element (156) extends for only a portion of the length of outer cannula (140). In such examples, one or more additional components such as arms and/or linkages can be used to facilitate transfer of movement from any suitable drive components to cutting element (156).

As best seen in FIG. 6, piercer (122) includes a generally hollow cylindrical wall (128) having a sharp distal tip (124). Cylindrical wall (128) defines a lumen (127) extending through the length of piercer (122) and one or more fluid openings (126) in communication with lumen (127). As will be described in greater detail below, distal tip (124) is generally configured to penetrate tissue of a patient when protruding from distal end (144) of outer cannula (140). As will also be described in greater detail below, lumen (127) and fluid openings (126) are generally configured to communicate fluid from piercer fluid source (164) into the interior of outer cannula (140).

As similarly described above with respect to needle assembly (20), needle assembly (120) of the present example is generally configured to provide variable dead space at various stages of a biopsy procedure to thereby permit access to suspicious lesions proximate sensitive anatomy. Generally, piercer (122) is configured to selectively move relative to outer cannula (140) to adjust or entirely eliminate the dead space of needle assembly (120) as defined by the distance between the end of distal tip (124) and distal end (144) of outer cannula (140). Consequently, piercer (122) can project from outer cannula (140) at some stages of a biopsy procedure and be retracted into outer cannula (140) at other stages of the biopsy procedure to provide variable dead space.

FIGS. 7A through 7D show an example of how such variable dead space can be used during a biopsy procedure. As best seen in FIG. 7A, piercer (122) is initially positioned relative to outer cannula (140) with distal tip (124) projecting from distal end (144) of outer cannula (140). In this configuration, the dead space of needle assembly (120) is generally increased relative to other configurations. As can be seen, while in this configuration, needle assembly (120) is used to penetrate tissue using distal tip (124) of piercer (122) to position distal end (144) of outer cannula (140) relatively close to a suspicious lesion. In other words, this configuration is used for course positioning of needle assembly (120). Optionally, in some uses, guidance of needle assembly (120) during penetration of tissue can be facilitated using various image guidance mechanisms such as x-ray guidance, ultrasound guidance, and/or optical coherence tomography (OCT) guidance.

During tissue penetration, cutting element (156) is generally in a retracted position and/or a straight configuration. In other words, cutting element (156) is generally straight and positioned between the outer surface of piercer (122) and the inner surface of outer cannula (140). Because cutting element (156) is positioned within outer cannula (140), this permits piercer (122) to both protrude from distal end (144) of outer cannula (140) and translate axially within outer cannula (140) without interference from cutting element (156).

After needle assembly (120) is positioned as described above with distal tip (124) of piercer (122) projecting from outer cannula (140), piercer (122) is retracted to reduce the dead space of needle assembly (120). As best seen in FIG. 7B, piercer (122) is retracted into the interior of outer cannula (140). In this position, piercer (122) is positioned laterally of internal lumen (148) and into internal recess (152) of projection (150). In the present example, such movement of piercer (122) is facilitated by piercer drive components (62) cannula drive components (66), or a combination thereof. Due to the decreased dead space in this configuration, needle assembly (120) is optionally positioned at this stage to position distal end (144) of outer cannula (140) closer to a suspicious lesion. In other words, this configuration is optionally used for fine positioning of needle assembly (120).

Once needle assembly (120) is positioned within tissue as desired, needle assembly (120) can be used to sever a tissue sample as shown in FIGS. 7B through 7D. To sever a tissue sample, tissue can be first prolapsed through distal end (144) of outer cannula (140) as shown in FIG. 7B such that issue is received within internal lumen (148) of outer cannula (140). The prolapse of tissue is controlled by vacuum applied to outer cannula (140), piercer (122), or both via fluid sources (64, 68).

Once tissue received within internal lumen (148), cutting element (156) is advanced distally as shown in FIG. 7C to sever a tissue sample. In particular, as cutting element (156) is advanced distally relative to outer cannula (140), portion of cutting element (156) is configured to bend transversely away from the inner surface of outer cannula (140) toward the opposite side of outer cannula (140). It should be understood, the particular location of the bend in cutting element (156) remains generally stationary while cutting element (156) continues to advance. Thus, a distal portion of cutting element (156) oriented at an angle relative to the inner surface of outer cannula (140) is advanced inwardly toward and through the prolapsed tissue to sever a tissue sample from the prolapsed tissue. In other words, cutting element (156) is configured to function similarly to a guillotine. As described above, such bending action can be facilitated by one or more features associated with cutting element (156) and/or outer cannula (140).

After the tissue sample is severed, the tissue sample is transported through outer cannula (140) as shown in FIG. 7D. As can be seen, vacuum is communicated through internal lumen (148) of outer cannula (140) to pull the tissue sample through internal lumen (148). As the tissue sample progresses through internal lumen (148), piercer (122) can communicate atmospheric air or pressurized fluid from piercer fluid sources (64) via lumen (127) and fluid openings (126). Such atmospheric air or pressurized fluid may be desirable to maintain a pressure imbalance between the proximal side of the tissue sample and the distal side of the tissue sample, thereby promoting transport of the tissue sample through internal lumen (148).

After the tissue sample is transported through outer cannula (140), the tissue sample can be deposited in a tissue sample holder (not shown) or other tissue collection area. The process described above with respect to FIGS. 7B through 7D can then be repeated without removal of needle assembly (120) from the patient to collect additional tissue samples until a desired number of tissue samples have been collected. Once a desired number of tissue samples are collected, needle assembly (120) can be removed from the patient.

### EXAMPLE COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A core needle biopsy device, comprising: a body; a needle assembly extending distally from the body and having a piercer, a hollow outer cannula, and a cutting element, the outer cannula defining an internal lumen, the piercer being disposed within the outer cannula, the cutting element extending along an outer surface or an inner surface of the outer cannula, the cutting element being configured to translate relative to the outer cannula; and a drive assembly configured to selectively move one or more of the piercer, the outer cannula, and the cutting element, the drive assembly being further configured to move the piercer relative to the outer cannula to open and close the internal lumen defined by the outer cannula.

### Example 2

The core needle biopsy device of Example 1, the outer cannula defines a projection protruding from a portion of the outer cannula to define an internal recess.

### Example 3

The core needle biopsy device of Example 2, the internal recess defined by the projection being disposed adjacent to the internal lumen defined by the outer cannula.

### Example 4

The core needle biopsy device of Examples 2 or 3, the internal recess being configured to receive the piercer.

### Example 5

The core needle biopsy device of any of Examples 2 through 4, the outer cannula further defining a lateral aperture, the projection being disposed on a side of the outer cannula opposite the lateral aperture.

### Example 6

The core needle biopsy device of any of Examples 1 through 5, the cutting element being configured to move independently relative to the piercer and the outer cannula to sever a tissue sample.

### Example 7

The core needle biopsy device of any of Examples 1 through 6, the cutting element being separate from the outer cannula and the piercer.

### Example 8

The core needle biopsy device of any of Examples 1 through 7, the piercer defining a sharp distal tip, the outer cannula defining an open distal end, the sharp distal tip of the piercer being configured to project from the open distal end of the outer cannula to penetrate tissue.

### Example 9

The core needle biopsy device of Example 8, the piercer further defining one or more fluid openings disposed distally of the sharp distal tip and a lumen extending through the piercer between the one or more fluid openings and a proximal end thereof, the one or more fluid openings being configured to communicate fluid with the internal lumen of the outer cannula.

### Example 10

The core needle biopsy device of Example 9, the one or more fluid openings being configured to communicate atmospheric air with the internal lumen of the outer cannula.

### Example 11

The core needle biopsy device of any of Examples 1 through 10, the cutting element being disposed on the outer surface of the outer cannula, the cutting element being configured to translate axially relative to the outer cannula to sever one or more tissue samples.

### Example 12

The core needle biopsy device of any of Examples 1 through 10, the cutting element being disposed on the inner surface of the outer cannula, the cutting element being configured to translate axially relative to the outer cannula to sever one or more tissue samples.

### Example 13

The core needle biopsy device of Example 12, the cutting element being configured to bend relative to the outer canula to sever the one or more tissue samples.

### Example 14

The core needle biopsy device of Example 12, the cutting element being configured to bend at a predetermined position along the length of the outer cannula to sever the one or more tissue samples.

### Example 15

The core needle biopsy device of Example 12, the cutting element being configured to bend transversely relative to the inner surface of the outer cannula at a predetermined position along the length of the outer cannula to sever the one or more tissue samples.

### Example 16

A needle assembly for use in a biopsy device, the needle assembly comprising: a hollow piercer having a sharp tip configured to penetrate, one or more fluid openings proximate the sharp tip, and a lumen extending from the one or more fluid openings to a proximal end of the piercer; an outer cannula disposed around the piercer, the outer cannula defining an internal lumen and an internal recess, the internal recess being disposed transversely of the internal lumen, the piercer being configured to move between the internal lumen and the internal recess to open and close the internal lumen; and a cutting element, the cutting element being configured to axially translate relative to the outer cannula to sever a tissue sample.

### Example 17

The needle assembly of Example 16, the outer cannula further defining an open distal end, the internal lumen being in communication with the open distal end, the internal recess being offset relative to the open distal end.

### Example 18

The needle assembly of Examples 16 or 17, the outer cannula including a substantially cylindrical outer surface, the outer cannula further including a projection projecting outwardly from the substantially cylindrical outer surface to define the internal recess.

### Example 19

The needle assembly of Example 18, a portion of the cutting element being configured to move transversely relative to a longitudinal axis defined by the outer cannula to slide the tissue sample.

### Example 20

A method for using a core needle biopsy device to collect multiple tissue samples using a single insertion of a needle assembly, the needle assembly including a piercer and an outer cannula around the piercer, the method comprising: inserting the needle assembly into tissue, a sharp distal tip of the piercer projecting from the outer cannula during insertion; retracting the piercer relative to the outer cannula to withdraw the sharp distal tip of the piercer into the outer cannula; further inserting the needle assembly into tissue after the step of retracting the piercer relative to the outer cannula; and moving a cutting element relative to the piercer and the outer cannula to sever a tissue sample.

### Example 21

The method of Example 20, the step of inserting the needle assembly into tissue being performed under optical coherence tomography (OCT) guidance.

### Example 22

The method of Examples 20 or 21, the step of further inserting the needle assembly into tissue further including moving a distal end of the outer cannula of the needle assembly proximate sensitive anatomy.

### Example 23

The method of any of Examples 20 through 22, the step of retracting the piercer relative to the outer cannula including reducing the dead space of the needle assembly.

### Example 24

The method of any of Examples 20 through 23, the step of retracting the piercer relative to the outer cannula being performed manually.

### Example 25

The method of any of Examples 20 through 24, the step of retracting the piercer relative to the outer cannula including moving the piercer transversely relative to the outer cannula.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

It should be understood that any of the versions of instruments described herein may include various other features in addition to or in lieu of those described above. By way of example only, any of the instruments described herein may also include one or more of the various features disclosed in any of the various references that are incorporated by reference herein. It should also be understood that the teachings herein may be readily applied to any of the instruments described in any of the other references cited herein, such that the teachings herein may be readily combined with the teachings of any of the references cited herein in numerous ways. Other types of instruments into which the teachings herein may be incorporated will be apparent to those of ordinary skill in the art.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

1. A core needle biopsy device, comprising:
(a) a body;
(b) a needle assembly extending distally from the body and having a piercer, a hollow outer cannula, and a cutting element, the outer cannula defining an internal lumen, the piercer being disposed within the outer cannula, the cutting element extending along an outer surface or an inner surface of the outer cannula, the cutting element being configured to translate relative to the outer cannula; and
(b) a drive assembly configured to selectively move one or more of the piercer, the outer cannula, and the cutting element, the drive assembly being further configured to move the piercer relative to the outer cannula to open and close the internal lumen defined by the outer cannula.

2. The core needle biopsy device of claim 1, the outer cannula defines a projection protruding from a portion of the outer cannula to define an internal recess.

3. The core needle biopsy device of claim 2, the internal recess defined by the projection being disposed adjacent to the internal lumen defined by the outer cannula.

4. The core needle biopsy device of claim 2, the internal recess being configured to receive the piercer.

5. The core needle biopsy device of claim 2, the outer cannula further defining a lateral aperture, the projection being disposed on a side of the outer cannula opposite the lateral aperture.

6. The core needle biopsy device of claim 1, the cutting element being configured to move independently relative to the piercer and the outer cannula to sever a tissue sample.

7. The core needle biopsy device of claim 1, the cutting element being separate from the outer cannula and the piercer.

8. The core needle biopsy device of claim 1, the piercer defining a sharp distal tip, the outer cannula defining an open distal end, the sharp distal tip of the piercer being configured to project from the open distal end of the outer cannula to penetrate tissue.

9. The core needle biopsy device of claim 8, the piercer further defining one or more fluid openings disposed distally of the sharp distal tip and a lumen extending through the piercer between the one or more fluid openings and a proximal end thereof, the one or more fluid openings being configured to communicate fluid with the internal lumen of the outer cannula.

10. The core needle biopsy device of claim 9, the one or more fluid openings being configured to communicate atmospheric air with the internal lumen of the outer cannula.

11. The core needle biopsy device of claim 1, the cutting element being disposed on the outer surface of the outer cannula, the cutting element being configured to translate axially relative to the outer cannula to sever one or more tissue samples.

12. The core needle biopsy device of claim 1, the cutting element being disposed on the inner surface of the outer cannula, the cutting element being configured to translate axially relative to the outer cannula to sever one or more tissue samples.

13. The core needle biopsy device of claim 12, the cutting element being configured to bend relative to the outer canula to sever the one or more tissue samples.

14. The core needle biopsy device of claim 12, the cutting element being configured to bend at a predetermined position along the length of the outer cannula to sever the one or more tissue samples.

15. The core needle biopsy device of claim 12, the cutting element being configured to bend transversely relative to the inner surface of the outer cannula at a predetermined position along the length of the outer cannula to sever the one or more tissue samples.

16. A needle assembly for use in a biopsy device, the needle assembly comprising:
(a) a hollow piercer having a sharp tip configured to penetrate, one or more fluid openings proximate the sharp tip, and a lumen extending from the one or more fluid openings to a proximal end of the piercer;
(b) an outer cannula disposed around the piercer, the outer cannula defining an internal lumen and an internal recess, the internal recess being disposed transversely of the internal lumen, the piercer being configured to move between the internal lumen and the internal recess to open and close the internal lumen; and
(c) a cutting element, the cutting element being configured to axially translate relative to the outer cannula to sever a tissue sample.

17. The needle assembly of claim 16, the outer cannula further defining an open distal end, the internal lumen being in communication with the open distal end, the internal recess being offset relative to the open distal end.

18. The needle assembly of claim 16, the outer cannula including a substantially cylindrical outer surface, the outer cannula further including a projection projecting outwardly from the substantially cylindrical outer surface to define the internal recess.

19. The needle assembly of claim 18, a portion of the cutting element being configured to move transversely relative to a longitudinal axis defined by the outer cannula to slide the tissue sample.

20. A method for using a core needle biopsy device to collect multiple tissue samples using a single insertion of a needle assembly, the needle assembly including a piercer and an outer cannula around the piercer, the method comprising:
(a) inserting the needle assembly into tissue, a sharp distal tip of the piercer projecting from the outer cannula during insertion;
(b) retracting the piercer relative to the outer cannula to withdraw the sharp distal tip of the piercer into the outer cannula;
(c) further inserting the needle assembly into tissue after the step of retracting the piercer relative to the outer cannula; and
(d) moving a cutting element relative to the piercer and the outer cannula to sever a tissue sample.

21.-25. (canceled)
